(19) United States
(12) Patent Application Publication
(10) Pub. No.: US 2005/0171486 A1
(43) Pub. Date: Aug. 4, 2005

(54) Safety syringe

(76) Inventor: Mark N. Hochman, Lake Success, NY (US)

Correspondence Address: GOTTLIEB RACKMAN & REISMAN PC, 270 MADISON AVENUE, 8TH FLOOR, NEW YORK, NY, 100160601, US

(21) Appl. No.: 11/095,369

(22) Filed: Mar. 31, 2005

Related U.S. Application Data
(63) Continuation-in-part of application No. 10/209,415, filed on Jul. 31, 2002.
Application No. 10/209,415 is a division of application No. 09/506,484, filed on Feb. 17, 2000, now Pat. No. 6,428,517.
(60) Provisional application No. 60/133,397, filed on May 10, 1999.
(60) Provisional application No. 60/173,374, filed on Dec. 28, 1999.

(51) Int. Cl. A61M 5/315

(52) U.S. Cl. 604/218

(57) ABSTRACT

A safety syringe is formed of an inner sleeve receiving a cartridge with liquid, an outer sleeve through which said inner sleeve is telescopically reciprocated, and a plunger assembly that is attached to the inner sleeve and used to eject the liquid. The needle is mounted on a front end of the inner sleeve and in a retracted position is wholly contained within the outer sleeve while in a protracted position the needle is exposed so that it can be used for an injection. Advantageously, in one embodiment, the inner sleeves can rotate with respect to each other causing the needle to rotate both when the needle is extended and when is retracted into the outer sleeve. Another feature of the invention is that in one embodiment the inner sleeve has an internal puncture member for puncturing the cartridge. Therefore, instead of a dental needle with two sharp tips, a standard needle with a single tip and a Luer connection may be used.

### RELATED APPLICATIONS

This application is a continuation-in-part of patent application Ser. No. 10/209,415 filed Jul. 31, 2002, which is a divisional application of patent application Ser. No. 09/506,484 filed Feb. 17, 2000 (now U.S. Pat. No. 6,428,517 issued Aug. 6, 2002) and which claimed priority to provisional applications Ser. No. 60/133,397 filed May 10, 1999 and Ser. No. 60/173,374 filed Dec. 28, 1999, all incorporated herein by reference.

### BACKGROUND OF THE INVENTION

A. Field of Invention

This invention pertains to a syringe used to inject a therapeutic fluid into human or animal tissues, and more particularly to a syringe that includes a needle movable along a longitudinal axis, which needle is optionally rotating about said axis as well. Importantly, prior to and immediately after the injection, the needle is disposed in a protective sleeve that prevents needle sticks. Preferably, after an injection is completed, a permanent locking mechanism is activated to trap the needle within the sleeve.

B. Description of the Prior Art

Traditional dental syringes are metal and designed to accommodate a double-ended needle on the threaded hub of the syringe. A pre-filled glass local anesthetic cartridge fits in the barrel of the syringe. The cartridge is pierced by a sharp working end of the needle that projects into the barrel of the syringe penetrating the diaphragm of the cartridge. It is often necessary for the dentist or other health care provider to provide multiple injections for a single patient throughout a dental procedure or appointment. The standard practice is to recap the needle after initial injection and then uncap and reuse the same needle on the one patient for subsequent injections. Frequently, the cartridge is changed, but the needle is rarely changed for a single patient. In certain situations however, the replacement of a needle may be necessary do to dulling of needle or the need for a different needle length or gauge. This then requires the removal and replacement of the needle. To accomplish this, the user recaps the needle, typically using a one-handed scoop technique or adaptive needle cap holder or add-on shield. The user must still manually manipulate the needle when removing it from the syringe, thus exposing the sharp end that protrudes into the anesthetic cartridge. This procedure provides a risk of percutaneous injury to users when handling anesthetic needles.

An important goal of dentistry and medicine in general is to insure that during any procedure involving interaction between a dentist or other care giver and a patient, utmost care is taken to protect the care giver from injury and/or infection with diseases. It has been recognized that the use of hypodermic needles, which activity is endemic in the field, is one particularly hazardous area. More particularly, the use of a hypodermic needle in dentistry and other medical fields has been shown to put the user at risk to inadvertent needle sticks, and presents a potential high risk to infection disease transmission for health care workers.

The use of hollow-core needles is the standard in drug delivery in medicine and dentistry. The use of the hollow-core needle dates back over 150 years when Charles Pravaz, a French general surgeon, patented such a device.

Currently several auto-retraction type syringes are known that provide increased safety by allowing the protruding needle˜to be retracted into a protective sheath after use. However, these systems where found to be unsatisfactory by the health care provider community. Other syringes are provided with removal caps. Some prior art syringes and needles with protectors are disclosed in the following U.S. patents: U.S. Pat. Nos. 5,120,310; 5,188,613; 5,267,961; 5,389,076; 5,423,758; 5,578,011; 5,632,733; 5,637,092; 5,810,775; 5,030,209; 4,911,693; 4,900,310; 4,813,426; 4,795,432; 4,664,654; 4,695,274; 4,966,592; 4,747,831; 4,900,311; 5,411,487; 5,106,379; 5,713,873.

Other systems are also known (e.g., U.S. Patent Application Publications 2002/0169421 and 2002/0068921 published Nov. 14, 2002 and Jun. 6, 2002, respectively) and U.S. Pat. Nos. 5,207,646 and 6,413,236. However, these systems are awkward to use and explicitly describes means for preventing needle rotation.

Generally, these prior art references have either one or more of the following deficiencies.

1) Hollow core needle that is rigidly affixed to the end of a syringe-, tubing or handle. The protruding needle presents a risk of inadvertent needle stick to the operator.

2) Existing auto-retracting syringes are difficult to use

3) Cannot be locked permanently to prevent reuse.

In the past 15 years regulatory agencies such as OSHA have attempted to improve the use and safety of needles in the health care industry. With the rise of infectious diseases, i.e., hepatitis and AIDS, protection of the health care workers has become a needed priority. It is supported in the medical/dental literature that inadvertent needle sticks represents a significant risk to our health care providers.

The proposed invention has been designed to eliminate the potential of inadvertent needle sticks after and during use.

For example, various medical organizations have suggested and several states have instituted rules for the protection of care givers while providing injections to patients. More specifically, rules have been promulgated requiring that injection needles on syringes be provided which can be easily removed prior to an injection and re-installed immediately after an injection.

Another related problem addressed by the present invention pertains to the deflection or bending of needles as they are inserted into the tissues of a patient. This deformation occurs because the needle is usually flexible because of its relatively small cross-sectional area and cannot resist effectively the axial and radial forces present during the insertion. This deflection is undesirable because it provides additional resistance during the insertion, to the movement of the needle and/or syringe, makes it difficult to guide the needle to a particular site preselected by the care giver. Moreover, if a needle is deflected, bent or otherwise deformed during insertion, it may cause more paint to the patient, trauma to the local tissues and other undesirable effect. It has been discovered (as disclosed in co-pending patent application Ser. No. 60/173,374 filed Dec. 28, 1999), incorporated herein by reference, that these disadvantages are eliminated or at least alleviated if the needle is rotated about its longitudinally about its axis as it is advanced along the axis into the patient tissues. Other syringes do not allow the use of a rotational insertion and removal technique to be used during a power assisted injection process.

### SUMMARY OF THE INVENTION

This syringe described herein overcomes the known disadvantages of existing syringes. It is a capable of both intermittent locking during use as well as permanent locking after use. In addition, an optional design configuration utilizes a single ended medical Luer-lock needle in combination with a standard local anesthetic cartridge enhancing the safety to the operator. An additional benefit of this device is an innovative design element in the barrel of the syringe that guides the needle into translation/rotation during injection, resulting in the reduction of force needed to penetrate the patient's tissue and also reduces needle deflection. Needle deflection in local anesthetic injections has been associated with increased failure rates. This same feature of simultaneous rotation/translation of needle will also result in a reduced amount of force to remove the needle from the patients' tissues, once again aiding in the safety and comfort to both the operator and patient. An auto-aspiration design is an innovative safety element in one of the potential designs of this device. The inclusion of this feature ensures greater safety for patients during the use of this device. Currently, no dental syringe is available with these features.

Because of the potential for nerve damage during dental anesthetic injections, it is desirable to have proper aspiration during certain injections. This helps prevent damage that may result in permanent loss of feeling and function in areas of the mouth, tongue and lip.

Because of its design, the subject syringe can be manufactured with fewer components then other syringes currently available in the marketplace.

Briefly, a safety syringe constructed in accordance with this invention includes two telescoping sleeves and a plunger assembly. Means are provided to selectively interlock the two sleeves in either a protracted and a retracted position. A cartridge filled with a liquid is disposed in the inner sleeve and is in fluid communication with a needle coupled to the inner sleeve. A user can advance the needle out of the outer sleeve . Preferably, as the needle is advanced it is rotated about its longitudinal axis at the same time for an angle of between 25 and 270 degrees. In one embodiment, the liquid is expressed as the needle is moving and rotating forward. In another embodiment the needle is advanced and rotated until it reaches the protracted position, and then the liquid is injected. In either case, at the end of the injection, the inner sleeve is withdrawn from the outer sleeve causing the needle to rotate as it retracted into the outer sleeve.

Once the needle is retracted into the second sleeve means provided on the outer sleeve are used to permanently capture the needle so that it cannot be removed.

In an alternate embodiment, means are provided that selectively urge the cartridge away from the needle thereby providing aspiration.

In an alternate embodiment, means are provided to puncture a membrane on the cartridge to provide access for the liquid. In this embodiment, a single ended standard medical needle can be used instead of a dental needle that possess a double ended needle with two sharp points.

### BRIEF DESCRIPTION OF THE FIGURES

FIG. 1 shows an orthogonal blown up view of a syringe constructed in accordance with this invention;

FIG. 2 shows an orthogonal view of the syringe of FIG. 1 with the needle protracted;

FIG. 3 shows an orthogonal view of the syringe of FIG. 1 with the needle extended;

FIG. 4 shows a side view of the push rod for the syringe of FIGS. 1-3;

FIG. 5 shows a side view of the push rod guide cap for the syringe of FIGS. 1-3;

FIGS. 6 and 6A show a side view of the inner sleeve for the syringe of FIGS. 1-3;

FIG. 7 shows a side view of the outer sleeve for the syringe of FIGS. 1-3;

FIG. 8 shows an enlarged partial cross-sectional view of outer sleeve of FIG. 7;

FIG. 8A shows a second enlarged partial cross-sectional view of the syringe taken along lines 8A-8A in FIG. 7;

FIG. 8B shows an enlarged partial top view of the outer sleeve of FIG. 7;

FIG. 9 shows an enlarged partial cross-sectional view of the syringe of FIGS. 1-3 with the inner sleeve being pushed into the outer sleeve;

FIG. 10 shows a cross-sectional view of the syringe in the protracted position;

FIG. 11 shows a partial cross-sectional view of the inner sleeve with a rim providing aspiration;

FIG. 12 shows a partial cross-sectional view of the inner sleeve with a coil spring providing aspiration; and

FIG. 13 shows a partial cross-sectional view of the inner sleeve with integral flexible fingers providing aspiration.

### DETAILED DESCRIPTION OF THE INVENTION

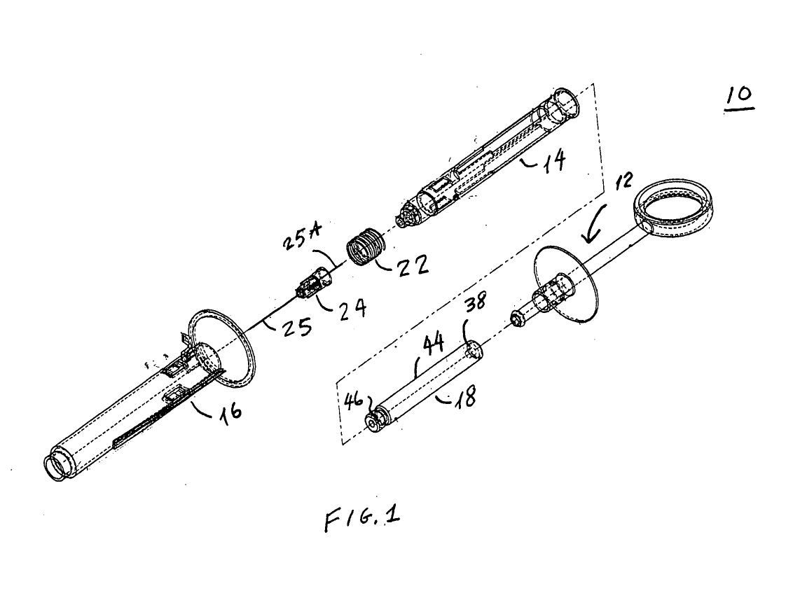

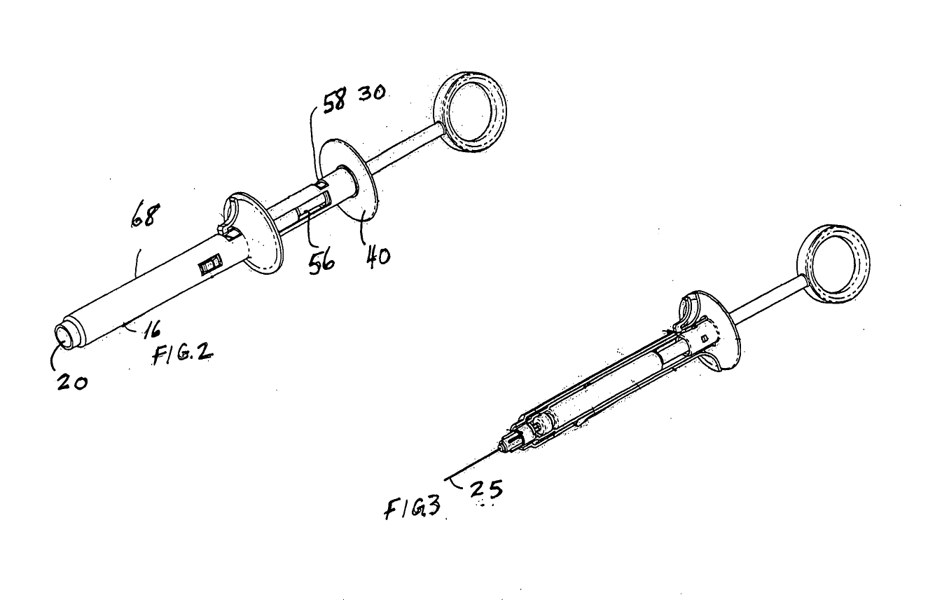

Referring first to FIGS. 1-10, a safety syringe 10 constructed in accordance with this invention includes a plunger assembly 12, an inner sleeve 14, and an outer sleeve 16 terminating at its distal end with an aperture 20. Briefly, a cartridge 18 filled with a desired liquid is inserted into the inner sleeve 14 and the inner sleeve 14 is the closed by inserting therein an end of the plunger assembly 12. A coil spring 22 is positioned on one end of the inner sleeve 12, a standard dental needle assembly 24 having a needle tip 25 is attached to the inner sleeve as shown in FIG. 2. The syringe 10 is then operated by forcing the inner sleeve 12 and its cartridge 18 through the outer sleeve 14 so that a portion of the needle assembly 24 exits through aperture 20. As the needle assembly exits through the aperture, the liquid within the cartridge is expressed through the needle and therefore the injection may be started as soon as the needle tip appears through the aperture. Preferably, as the needle tip 25 is advancing toward its final position shown in FIG. 3, it is simultaneously rotated about its longitudinal axis.

After the injection is complete, the inner sleeve can be released and the coil spring 22 causes the needle assembly 24 to be rapidly withdrawn into the outer sleeve 14. The syringe can be reused, or alternatively, a permanent lock mechanism can be activated to trap the needle assembly 24 within the outer sleeve 16. The whole syringe 10 can then be discarded safely since the needle tip 25 is not exposed. Details of the syringe elements and how they interact to provide the described functions are provided below.

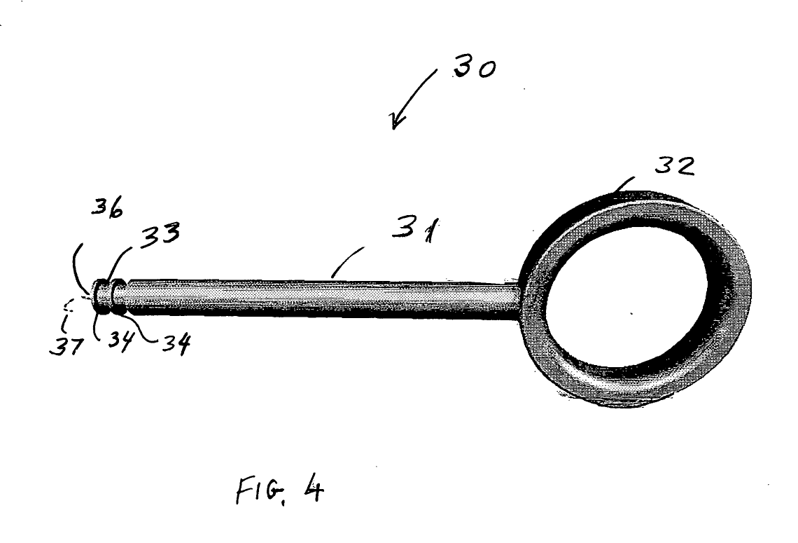

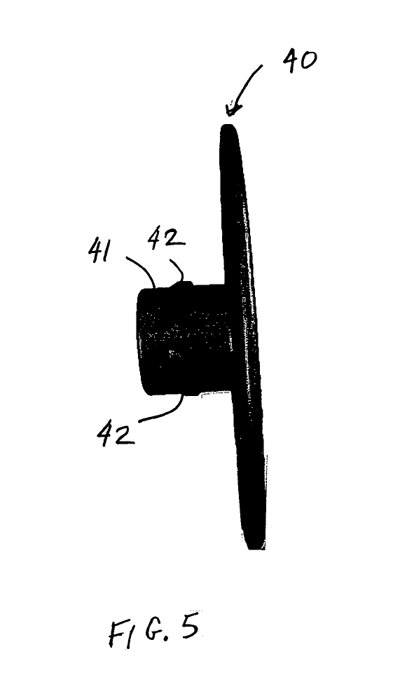

The plunger assembly 12 consists of an elongated push rod 30 (shown in FIG. 4) and a guide cap 40 (shown in FIG. 5). Push rod 30 has a shaft 31 attached at one end to a finger ring 32 or other similar means for engaging the thumb or finger of a health care provider. At the opposite end, shaft 31 has a reduced diameter portion 33 formed with preferably two O-rings 34. The end face 36 may be provided with a hook or a barb 37. The two O-ring design is somewhat preferable because it provides a suitable frictional grip and therereby the following advantages:
1. Two O-ring design allows the plunger to be properly stabilized within the anesthetic cartridge at two axially spaced contact points within the glass cartridge. With a single O-ring design the plunger may pivot within the inner surface of the glass cartridge making the plunger unstable during use.
2. The two O-ring design provides increased frictional drag of the plunger as it is moved within the glass cartridge. This serves 2 additional important purposes:
   a. The increased drag of the two O-rings ensures a slower rate of delivery because of the increased friction force encountered against the glass cartridge while depressing the plunger thus ensuring that the anesthetic solution will be expressed at a slower rate producing a more comfortable injection for the patient. It is well documented in the dental and medical literature that a slower injection is a more comfortable injection for the patient.
   b. The increased drag of the two O-ring design enables the glass cartridge to be removed simultaneously with the plunger assembly as the plunger assembly is disconnected or withdrawn from the inner cylinder. This can add in the efficiency of removal or replacing the cartridge at the end of the injection.

The design of the plunger is not limited to a two o-ring design and may use more or less number of O-rings to accomplish the same objectives. It is also conceivable that another type of “gasket component” could be used such as a flexible collar that is made from a soft material such as silicone, but not limited to silicone, to achieve a similar outcome.

Between its two ends, rod 30 supports the push rod cap 40 shown in more detail in FIG. 5. The cap 40 is formed with a short sleeve 41 formed with two snap tabs 42. Cap 40 can be glued to the rod 30, can be held in place by two O-rings (not shown) or the push rod 30 and the cap 40 can be molded unitarily.

As discussed above, the cartridge 18 holds a desired liquid such as an anesthetic. Typically, such a cartridge has a cylindrical glass or plastic sidewall 44 (FIG. 1), with a rubber piston 38 at one end, and a rubber membrane 46 encased in a metal sheath at the other end.

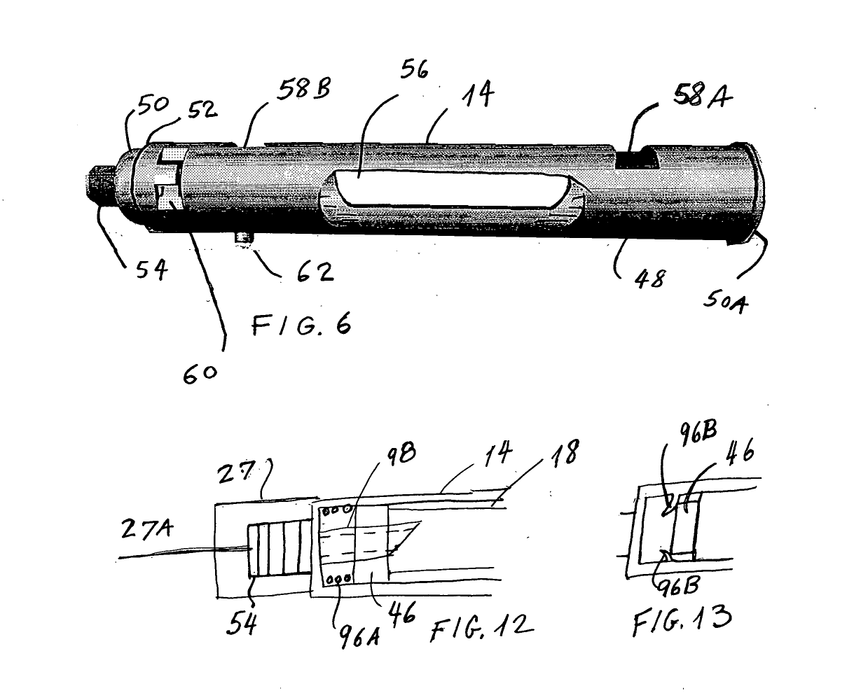

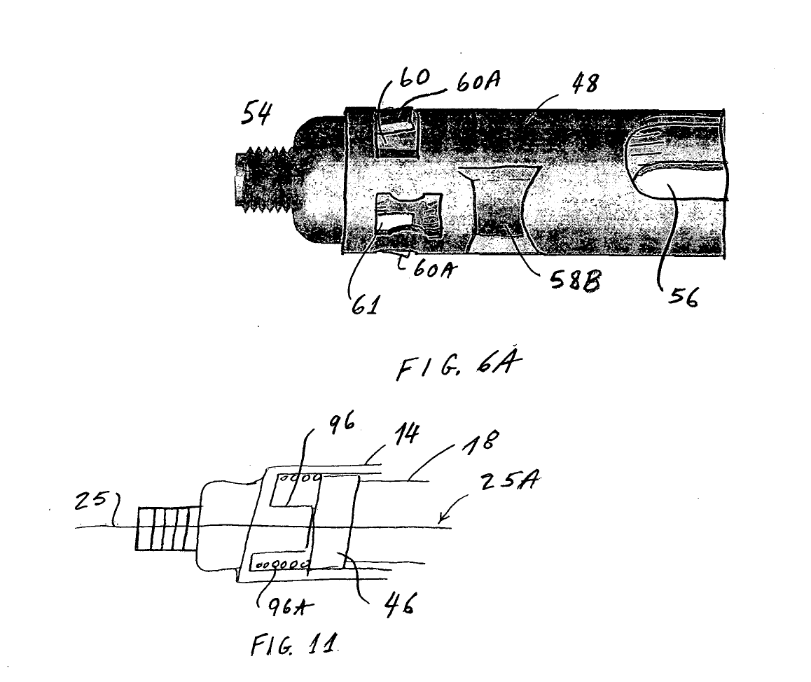

The inner sleeve 14 shown in detail in FIGS. 6 and 6A is formed with a cylindrical sidewall 48 with an open mouth 50A at one end, and a closure 50 at the other end. The closure 50 includes a shoulder 52 surrounding a hub 54. The cylindrical wall 48 is formed with one or more elongated (preferably two) windows 56. In addition, the wall 48 with two similarly shaped transversal openings 58A, 58B, one near each end of sleeve 14. The two openings 58A, 58B are angularly offset from each other. The sleeve 14 is further provided with two additional openings or depressions 60 spaced peripherally about wall 48, preferably opposite to each other. These openings 60 are formed with a flexible tongue 60A. As seen in FIG. 6A, the tongues 60A are formed so that their ends normally extend outwardly of the surface 48. Between openings or depressions 60 there is butterfly-shaped opening 61. Wall 48 is also formed with a pin 62.

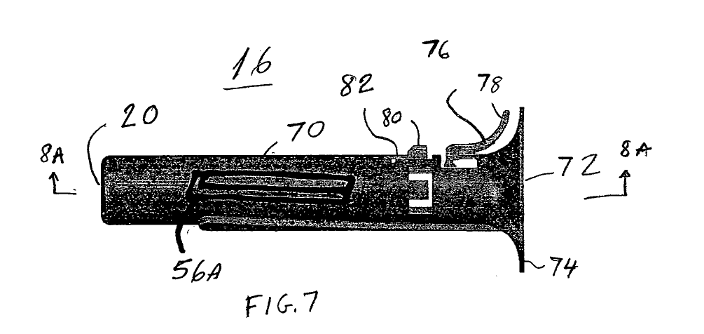

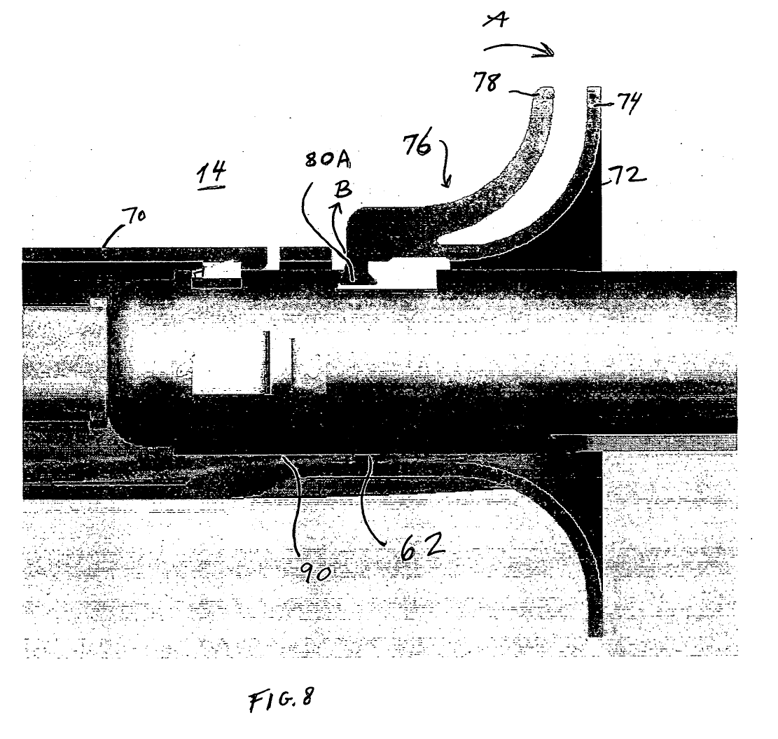

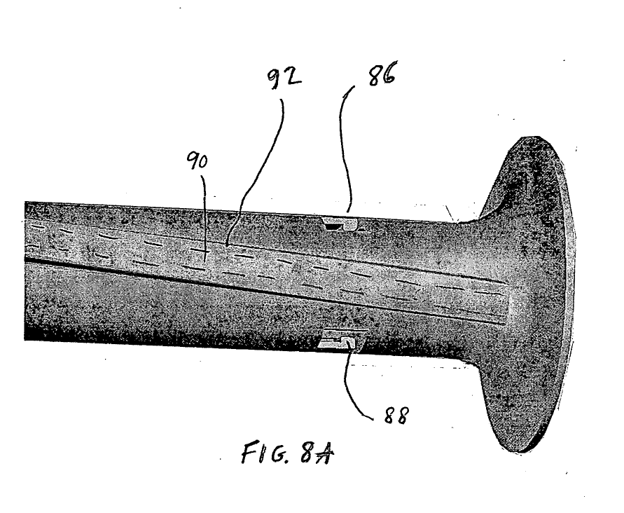

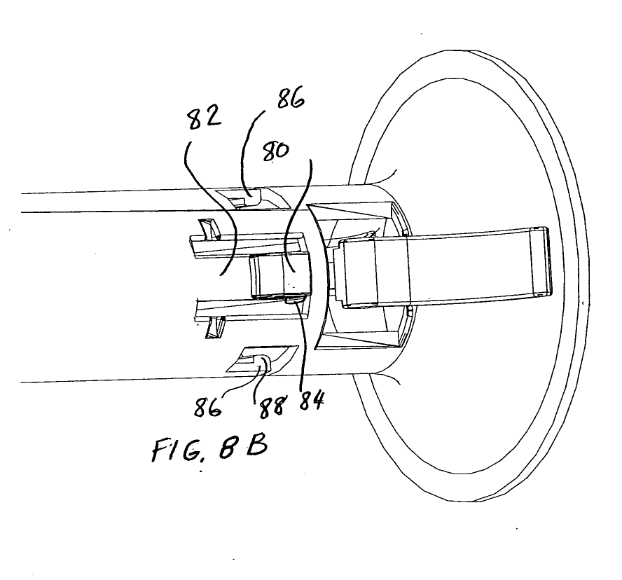

Referring to FIGS. 7, 8, and 8A, the outer sleeve 16 is formed with a cylindrical outer wall 70 with a mouth 72 at one end and opening 20 at the other. A finger collar 74 is formed around mouth 72 having about the same diameter as the push rod cap 40. Adjacent to collar 74 there is a control lever 76. The control lever 76 includes a curved arm 78 shaped and sized to be operated with a finger (like the trigger of a pistol) and a tong 80 extending inside the outer sleeve 16. The control lever 76 is preferably molded unitarily with the outer wall 70 and collar 74 in such a manner that it can be slightly pivoted when pulled by a finger as indicated by arrow A to cause the tong 80A to rise outwardly of the sleeve 14, as indicated by arrow B. When the arm 78 is released, the tong moves back to the position show in FIG. 8. As seen in FIGS. 7 and 8B, axially spaced from the control lever 76 there is a tab 80. This tab 80 is supported by a frangible strip 82 attached to the sidewall 70 and includes a tooth 84 extending into the sleeve 16. Two or more tabs 80 may also be provided.

Optionally, the sidewall 70 can be formed with two elongated windows windows 56A having generally the same size and shape as windows 56 on the inner sleeve. The purpose of the two sets of coinciding windows 56, 56A is as follows:
1. The oral cavity is a moist and humid environment and it is possible that the humidity encountered by the device may result in a fogging effect of the materials used thus hampering the ability of the user to see the cartridge 18 contained within the syringe assembly 10. However, in some instances (for example, aspiration) it is important that user be able to see the contents of the cartridge 18. The two set of elongated windows 56, 56A are conincident to ensures that proper ventilation is provided to prevent the fogging or clouding when being used.
2. In addition, the windows allows the sleeves to be molded of a material that is not translucent and is relatively opaque but still enables the user to see cartridge 18
3. The windows may be used to remove the cartridge from the sleeve 14.

Advantageously, sleeve 16 also includes two fingers 86. Each finger 86 includes a respective inwardly extending projection 88 as shown in FIGS. 8A and 8B.

The sleeve 16 also includes a helical channel 90. The channel is disposed on the inner surface of sleeve 16 and is sized and shaped to receive pin 62. Preferably, the sleeve 18 includes a rib 92 forming channel 90, although, if the sleeve 16 is thick enough, rib 92 may not be necessary. The sleeve 16 is preferably made from a molded plastic material.

Returning to FIGS. 1-3, initially, the syringe 10 may be supplied in two parts: sleeves 14 and 16 form one part, the plunger assembly 12 forms another part and the health care provider supplies the needle and the cartridge. The sleeve 14 is disposed in the protracted position of FIG. 3, so that it is essentially all the way inside sleeve 16. The two sleeve are being biased by the spring 22 is compressed between the two sleeves, however they are maintained in this protracted position by the tongue 80A being disposed in hole 58A and thereby engaging the inner sleeve.

A user applies an injection to a patient using the syringe 10 as follows. First, he attaches an appropriate needle 24 by mounting on hub 54. In this position, the needle tip 25 extends outwardly, as shown in FIG. 2, while the inner needle end 25A extends inside the sleeve 14. Next, the user pulls the lever 76 causing the tongue 80A to disengage from the sleeve 14. As a result, the spring 22 causes the sleeve 14 outward to the retracted position and shifting the needle into the sleeve 16 so that it is no longer exposed. Sleeve 14 moves back until the tongues 60A come into contact with the inwardly extending projections 88 on fingers 86. Thus, tongues 60A, fingers 86 and projections 88 are slightly flexible to allow the sleeve 14 to be inserted into sleeve 16, but once the sleeve 14 is inserted, these elements cooperate to form a stop that prevents the sleeve 14 to be removed from sleeve 16. Moreover, the control lever 76 also engages the sides of opening 58B thereby capturing sleeve 14 in the retracted position.

Next, the user inserts a cartridge 18 into sleeve 14. The user pushes the cartridge all the way, causing the needle end 25A to penetrate the membrane 46 and is immersed in the liquid contained therein.

Next, the user inserts the plunger assembly into the sleeves. More particularly, he pushes the rod 30 into the sleeve 14. The rod 30 and the sleeve 14 are dimensioned so that as the end 36 of rod 30 comes into contact with the piston 38, the inner surface of the sleeve 14 telescopically fits over short sleeve 41 and a frictional engagement is created between the sleeve 14 and the snap tabs 42. The assembled syringe is shown in FIGS. 2 and 8. If the rod 30 has a barb 37, the barb penetrates and engages the piston 38.

The syringe could be used in two modes. In one mode, the user holds the syringe with the thumb extending through ring 32 and two fingers trained around finger collar 74. One finger is used to release the sleeve 14 by pushing control lever 76. The user then squeezes the syringe between his fingers and thumb together in a normal fashion. As the user is squeezing the syringe three events occur simultaneously. First, the piston 38 is pushed by rod 30 into the cartridge 18 causing the liquid to be expressed through needle 25. Second, the sleeve 14 with cartridge 18 starts moving forward so that the needle starts extending outwardly of aperture 20. Once a substantial portion of the needle tip 25A is visible, the injection can be started. Third, as discussed above, the pin 62 is disposed in groove 90 so that as the sleeve 14 advances through sleeve 16 groove 90 forces the sleeve 14 and needle 24 through pin 62 to rotated about their common longitudinal axis. In other words, the needle 24 is translated and rotated simultaneously while the liquid is ejected and injected. This composite motion continues until the sleeve 14 reaches it initial protracted position. At this position, the control lever 76 again engages the sleeve 14 through opening 58A. FIG. 9 shows an intermediate position with the plunger assembly 12 and sleeve 14 advancing, and FIG. 10 shows the syringe during this final, intermediate locked position. Once this position is reached, the user can just continue injecting the patient. As disclosed in my earlier applications, this three-fold action is very advantageous because it reduces the pain normally associated with the injection, reduces tissue damage, and so forth.

Once the injection is complete, the user stops squeezing the syringe and activates the control lever 76 again. Once again, the needle 24 jumps back into the outer sleeve. Because pin 62 still engages groove 90, during this motion, the needle is still caused to translate and rotate simultaneously thereby reducing pain and trauma, and also reducing unnecessary tissue damage. The user can now remove the plunger assembly and the spent cartridge, replace the cartridge in the inner sleeve and start a new injection. This operation is facilitated by the fact that there are one or more (preferably two) O-rings 34 engage the sidewall of the cartridge and stabilize the plunger assembly as it is being withdrawn from the inner sleeve, and when the plunger assembly is removed, the cartridge is pulled out and discarded at the same time. However, if it becomes too difficult to remove the cartridge, the user can remove the cartridge by hand by pushing it axially through windows 56.

After the injection is completed, the plunger assembly is discarded together with the spent cartridge. As discussed above, the control lever 76 is used to selectively and temporarily lock sleeve 14 in either the retracted or protracted positions. However, recently some authorities have recommended or mandated that the needle should be immobilized after one use so that it and the syringe cannot be reused. In the present invention, after the sleeve 14 is pushed into the retracted position, tab 80 is pushed inward causing it to bend and engage the sidewalls of hole 61. The frangible strip 82 is permanently deformed by this action so that it is no longer flexible and cannot be used to retract tab 80. This tab 80 permanently engages hole 61 and does not allow the sleeve 14 to be shifted or removed from sleeve 16. In this manner, the needle 24 is captured within sleeve 16.

In a second mode of operation, the user first squeezes the push rod cap 40 and the finger collar 74 together. This action causes the inner sleeve to move into the outer sleeve, as described above, without the liquid being injected. When the inner sleeve reaches the Protracted position (FIG. 3), the needle can then be inserted into a patient tissue and liquid can be injected in the normal manner. After the injection is completed, the needle can be retracted into the outer sleeve, as described above.

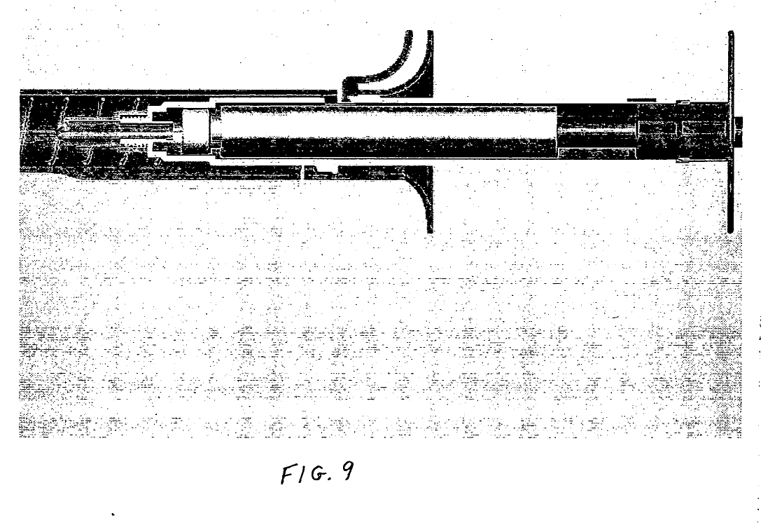

In some instances, it may be recommended that the syringe be aspirated at the beginning of the injection. For this purpose, in one embodiment of the invention, shown in FIG. 11, the inner sleeve is provided with an annular rim 96 on the end wall of sleeve 14 positioned adjacent to the membrane 46. During injection, the cartridge 18 is pushed forward so that the membrane 16 bears against rim 96. The membrane is soft and resilient and therefore it is deformed by the rim 96. If aspiration is desired, the user releases the finger ring and the rim pushes the membrane 46 and cartridge 18 toward the right, away from needle 24. This action causes the syringe to aspirate some blood or other body fluid. When injection is resumed, the membrane 46 is pushed back, against the rim 96. In an alternate embodiment of the invention, instead of, or in addition to the rim 96, the sleeve 14 is provided with a coil spring 96A. Again, if the finger ring 32 is released, the spring 96A causes the cartridge 18 to pull back and aspirate. In another embodiment shown in FIG. 13 the inner sleeve 14 is formed or molded with integral flexible fingers 96B on its inner wall as shown. The fingers 96B are bent forward by the cartridge 18 while the plunger assembly is advanced. When the plunger assembly is released, the fingers spring back, causing the cartridge to move backwards, away from front end of the inner sleeve 12.

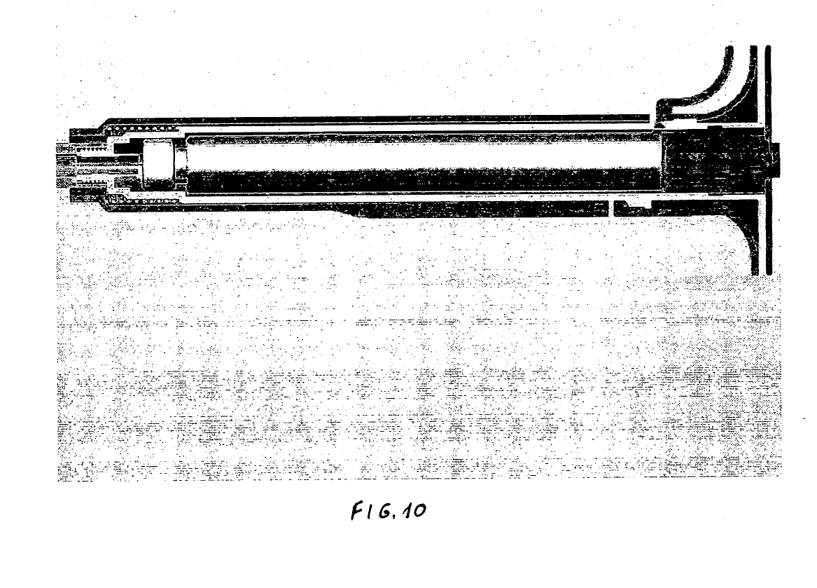

In another advantageous embodiment shown in FIG. 12, the sleeve 14 is provided on its end wall with a hollow spike 98. The spike is sized and shaped to penetrate the membrane 46 when the cartridge 18 is pushed into the sleeve 14. The inner lumen of the spike is in communication with the hub 54. In this embodiment, instead of using a dental needle with two needle tips, a needle assembly 27 can be used with a single hollow needle 27A that receives the liquid from cartridge 18 through spike 98. Again, the needle assembly 27 is provided with a Luer lock so that it can be readily mounted onto the hub 54. Thus, the needle 24 could be a standard needle.

While it is preferable to have the needle rotate as it is advanced out of the outer sleeve and retracted back into the outer sleeve, obviously safety syringe can be modified to eliminate this rotation, for example, by omitting the pin 72, the groove 90, or both.

Obviously, the safety syringe provides numerous advantages not found in the prior art. More specifically, the safety syringe prevents or at least reduces the sharps injuries from exposed needles. Moreover, by reducing or eliminating exposure to sharp needles, the safety syringe also reduces the risk to exposure to blood and other potentially infections substances. The syringe meets the requirements set forth for this purpose by various state and federal agencies for preventing sharps injuries and exposure to potentially contaminated or infectious substances.

The syringe is highly innovative and includes many desirable characteristics not currently available in other syringes, in the dental or medical market place. These features include a smaller and compact size, one-handed operation from a single position on the device, reduced needle insertion force, reduced needle deflection, integrated aspiration function, selectively intermittent or permanent locking the needle within the syringe. These features provide the following advantages:

a. The compact design insures that the syringe can be used by health care providers having various hand-sizes;

b. One-handed operation insures that that syringe is operated reliably and consistently;

c. Multiple injections can be performed on the same patient safely, without requiring the recaping of the needle in between injections;

d. The intermittent locking reduces the risk of accidental needle stick during normal use;

e. Permanent locking insures that the needle can be safely disposed while it is completely encapsulated and captured within the outer sleeve.

At least one study has shown that at the present health care practitioners are reluctant to use other types of safety syringes because these syringes lack the following features and therefore they are more difficult and less convenient to use:
Needle visibility;
Cartridge visibility;
Difficulty in breaking down and disposing after use;
Difficulty in replacing needle during multiple injections.

Moreover the study has also shown that the following characteristics are desirable for a safety needle:
Passivity (little or no user manipulation required to activate safety feature)
Reliability of the safety feature;
Ability to exchange the cartridge quickly and efficiently;
Visibility of the needle tip and cartridge.
Adaptability to various hand sizes
Easy and safe breakdown;
Cost savings.

The safety syringe contains all these desirable characteristics.

Numerous modifications may be made to this invention without departing from its scope as defined in the appended claims.

1. A safety syringe comprising:
a plunger assembly including a rod;
an inner sleeve sized and shaped to receive a cartridge with liquid and said plunger assembly, said plunger assembly being adapted to force the liquid from said cartridge when said rod is moved with respect to said inner sleeve, said inner sleeve having a front end adapted to receive a needle, said needle being in communication with said cartridge to receive said liquid;
an outer sleeve telescopically receiving said inner sleeve; and
a stop mechanism adapted to permanently retain said inner sleeve within said outer sleeve while allowing said inner sleeve to slide telescopically within said outer sleeve.

2. The safety syringe of claim 1 further comprising a first intermittent lock means for locking said inner and outer sleeves in a first position and a second intermittent lock means for locking said inner and outer sleeve in a second position, said sleeves being prevented from moving telescopically with respect to each other when one of said first and second lock means is active.

3. The safety syringe of claim 2 wherein said lock means includes a pivoting lever disposed on said outer sleeve and respective first and second lever engagement members associated with said inner sleeve.

4. The safety syringe of claim 1 wherein said outer sleeve is formed with a circumferential collar.

5. The safety syringe of claim 4 wherein said rod further includes a finger ring, said finger ring cooperating with said collar to cause said rod to eject liquid from said cartridge through said needle when said finger ring and collar are manually squeezed together.

6. The safety ring of claim 1 wherein said rod has a rod end received in said cartridge for ejecting said liquid, and at least O-ring mounted on said rod end and arranged to stabilize said rod end as it reciprocates through said cartridge.

7. A safety syringe comprising:
a plunger assembly including a rod;
an inner sleeve with a front end adapted to receive a needle and a rear end with a mouth for receiving a cartridge with a liquid, said liquid being ejected through said needle when said rod is inserted into said cartridge within said inner sleeve;
an outer sleeve cooperating with said inner sleeve to define a retracted and a protracted position, wherein said needle is exposed in said protracted position and is fully contained within said outer sleeve in said retracted position;
an actuating mechanism telescopically and selectively moving said inner sleeve with respect to said outer sleeve between protracted and retracted positions; and
a rotation mechanism simultaneously rotating said inner and outer sleeves with respect to each other as said inner and outer sleeves move between said positions, said needle rotating with said inner sleeve.

8. The syringe of claim 7 further comprising a spring urging said sleeves toward said protracted position.

9. The syringe of claim 7 further comprising a first lock intermittently locking said sleeves in said protracted position, and a second lock intermittently locking said sleeves in said retracted position.

10. The syringe of claim 9 wherein said first and second lock includes a lever disposed on said outer sleeve and first and second lever engaging holes formed in said inner sleeve.

11. The syringe of claim 10 further comprising a spring urging said sleeves toward said retracted position, wherein said sleeves are moved automatically to said protracted position, when said sleeves are in said protracted position and said lever is released.

12. The syringe of claim 7 further comprising a permanent lock that when activated immobilizes said needle within said outer sleeve.

13. The syringe of claim 12 wherein said permanent lock includes a tab formed on said outer sleeve and a frangible member that deforms when said tab is pressed to cause said tab to permanently engage said inner sleeve.

14. A safety syringe comprising:
a plunger assembly including a rod;
an inner sleeve with a front end adapted to receive a needle and a rear end with a mouth for receiving a cartridge with a liquid, said liquid being ejected through said needle when said rod is inserted into said cartridge within said inner sleeve;
an outer sleeve cooperating with said inner sleeve to define a retracted and a protracted position, wherein said needle is exposed in said protracted position and is fully contained within said outer sleeve in said retracted position;
an actuating mechanism telescopically and selectively moving said inner sleeve with respect to said outer sleeve between protracted and retracted positions; and
an aspiration mechanism automatically providing aspiration when said actuating mechanism is released.

15. The syringe of claim 14 wherein said aspiration mechanism includes a flexible member that urges said cartridge away from said front end.

16. The syringe of claim 15 wherein said flexible member is coil spring.

17. The syringe of claim 15 wherein said flexible member is a plastic member formed integrally in said inner sleeve.

18. The syringe of claim 15 wherein said sleeves are formed with elongated windows arranged and constructed to allow cartridge removal, venting and direct viewing of the cartridge contained therein.

19. A safety syringe comprising:
a plunger assembly including a rod;
an inner sleeve with a front end adapted to receive a needle and a rear end with a mouth for receiving a cartridge with a liquid, said liquid being ejected through said needle when said rod is inserted into said cartridge within said inner sleeve, said inner sleeve including an inner member arranged to puncture said cartridge when said cartridge is inserted into inner sleeve, said inner sleeve further including a threaded member disposed at said front end to receive a needle, said threaded member and said puncture member cooperating to provide liquid from said cartridge to said needle without said needle extending into said inner sleeve;
an outer sleeve cooperating with said inner sleeve to define a retracted and a protracted position, wherein said needle is exposed in said protracted position and is fully contained within said outer sleeve in said retracted position; and
an actuating mechanism telescopically and selectively moving said inner sleeve with respect to said outer sleeve between protracted and retracted positions.

20. The syringe of claim 19 wherein said puncture member is a hollow spike.

21. The syringe of claim 19 wherein said threaded member is a Luer lock adapted to accept a needle with matching Luer connector.
